(19) United States
(12) Patent Application Publication
(10) Pub. No.: US 2008/0147123 A1
(43) Pub. Date: Jun. 19, 2008

(54) Occipital plate assembly

(75) Inventor: Leah Schermerhorn, San Diego, CA (US)

Correspondence Address: MCDERMOTT, WILL & EMERY, 4370 LA JOLLA VILLAGE DRIVE, SUITE 700, SAN DIEGO, CA, 92122, US

(73) Assignee: SeaSpine, Inc.

(21) Appl. No.: 11/639,469

(22) Filed: Dec. 14, 2006

(51) Int. Cl. A61B 17/70

(52) U.S. Cl. 606/278

(57) ABSTRACT

An occipital plate assembly is provided with extension posts on which rod housings are slidably and angularly mounted. The rod housings are slidable in the transmedial-lateral direction to accommodate variation and distance between fixation rods, and are angularly adjustable in the sagittal plane, thereby accommodating rods at varying angles.

### FIELD

The present disclosure relates generally to spinal implants, more specifically to a spinal implant for use in the occipital-cervical region of the spine, including an occipital plate for attachments to the lower skull.

### BACKGROUND

Arrangements have been provided for implantation in the spine, these arrangements generally including a series of bone fasteners, such as hooks or screws, that are secured to the vertebrae, and which are used to hold stabilizers, such as a rod or a plate that spans vertebrae for stabilization, fixation and/or alignment of the vertebrae.

Typically, a spinal rod assembly includes two sets of rods that are fixed to adjacent vertebrae on either side of the spinous process to span a section of spine. The bone anchors may include a number of fixation devices, such as screws or hooks, that are used for fixation to the spine, and anchors such as rod anchors that secure the rods to the fixation devices. In some of these systems, the component parts are a single integral unit, while other systems utilize assembled components.

Systems have been provided in which a unitary plate and rod system is bent in two planes in order to properly adjust the positioning with respect to the occiput. Such devices provide for a limited flexibility of installation by the surgeon, as bending of the rod and plate system in two planes is relatively difficult to do to achieve a precise fit.

### SUMMARY

There is a need for an occipital plate assembly which provides greater flexibility of installation to a surgeon.

This and other needs are met by embodiments of the present disclosure which provide an occipital plate assembly comprising a center plate configured for connection to a skull, and extension posts extending outwardly from the center plate. A rod housing is rotatably mounted on each of the extension posts, the rod housings having an opening configured to receive a rod.

The earlier stated need and others are also met by other embodiments of an occipital plate assembly, which comprise a center plate configured for attachment to an occiput, and rod housings coupled to the center plate. The rod housings are configured for securing fixation rods to the center plate. The rod housings are angularly adjustable in a sagittal plane with respect to the center plate.

The foregoing and other features, aspects and advantages of the disclosed embodiments will become more apparent from the following detailed description and the accompanying drawings.

### BRIEF DESCRIPTION OF THE DRAWINGS

FIG. 1 is a top perspective view of a portion of an occipital plate assembly constructed in accordance with the disclosed embodiments.

FIG. 2 is a top view of the occipital plate assembly portion of FIG. 1.

FIG. 3 is a top view of a center plate of the occipital plate assembly of FIG. 1.

FIG. 4 is a side view of the center plate taken in the direction of arrow A of FIG. 3.

FIG. 5 is a front view of the occipital plate assembly of FIG. 3 taken in the direction of arrow B.

FIG. 6 is a perspective view of a rod housing constructed in accordance with disclosed embodiments, depicted in isolation.

FIG. 7 shows a side view of the occipital plate assembly portion of FIG. 1, with an illustration of angular adjustability of the rod housing with respect to the center plate.

FIG. 8 is a perspective view of a locking cap in isolation, constructed in accordance with embodiments of the present disclosure.

FIGS. 9 and 10 depict views of the occipital plate assembly with rods attached to the center plate by the locking cap in accordance with disclosed embodiments.

### DETAILED DESCRIPTION

The disclosed embodiments address and solve problems related to occipital plate assemblies. In particular, the disclosed embodiments provide for greater flexibility and adjustment so that a surgeon may more properly and easily fit an occipital plate assembly to a patient during surgery. This is achieved, in part, by the disclosed embodiments which provide an occipital plate assembly comprising a center plate configured for attachment to an occiput, and angularly adjustable rod housings coupled to the center plate. These rod housings are configured for securing fixation rods to the center plate. The angular adjustability of the rod housings in a sagittal plane with respect to the center plate accommodate rods that are bent at varying angles and eliminate the need for additional bending of the rods. This adjustability greatly increases the flexibility provided to the surgeon for implantation of the assembly and the fixation rods.

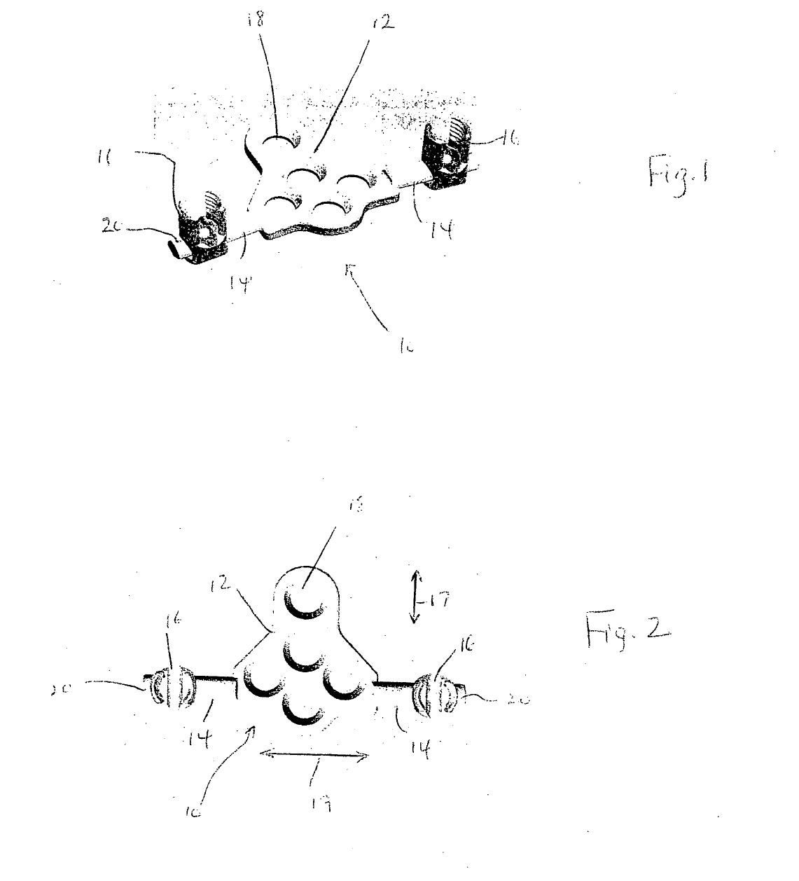

FIG. 1 is a perspective view of an occipital plate assembly 10 constructed in accordance with disclosed embodiments. Similarly, FIG. 2 is a top view of the occipital plate assembly 10 of FIG. 1. The occipital plate assembly 10 includes a center plate 12 which is configured to be attached to the occiput of a skull. For this purpose, a plurality of holes 18 are provided through which fasteners, such as occipital screws (not shown in FIGS. 1 and 2) are received to firmly attach the center plate 12 to the occiput. The size and shape of the center plate 12 may be different in different embodiments. For example, the sizing of the center plate 12 may be different to accommodate different size occiputs and shapes. A different number of holes 18 may be provided, dependent upon the size and shape of the center plate 12.

In the embodiments of FIGS. 1 and 2, the holes 18 extend in the vertical direction 17, which may also be termed a cephalad-caudal direction. Two of the holes 18 are also provided in a transmedial-lateral direction 19. The number and configuration of screw holes 18 are therefore varied in different embodiments.

The center plate 12 includes two extension posts 14 that extend outwardly from the center plate 12. In the embodiment of FIGS. 1 and 2, the extension posts 14 extend directly along the transmedial-lateral direction. In other embodiments (not illustrated), the extension posts 14 are angled relative to the transmedial-lateral direction 19. The occipital plate assembly 10 in such embodiments are therefore shaped more like a Y-shape, rather than the illustrated T-shape. In certain preferred embodiments, the extension posts 14 are integrally formed with the center plate 12. In other embodiments, the extension posts 14 are attached to the center plate 12 by any suitable method that provide secure and non-rotatable attachment.

Each extension post 14 carries a rod housing 16. Each rod housing 16 is slidable along the transmedial-lateral direction 19 on one of the extension posts 14. A pin 20, which may be inserted into the distal ends of extension posts 14 following a mounting of the rod housing 16 on the extension posts 14, acts as a retaining element to retain the rod housings 16 on the extension posts 14. The occipital plate assembly 10 can therefore be handled as a one-piece assembly, thereby facilitating handling for the surgeon during an implantation process, rather than requiring mounting of the rod housings 16 on the extension posts 14 during a surgical procedure or otherwise trying to hold them on.

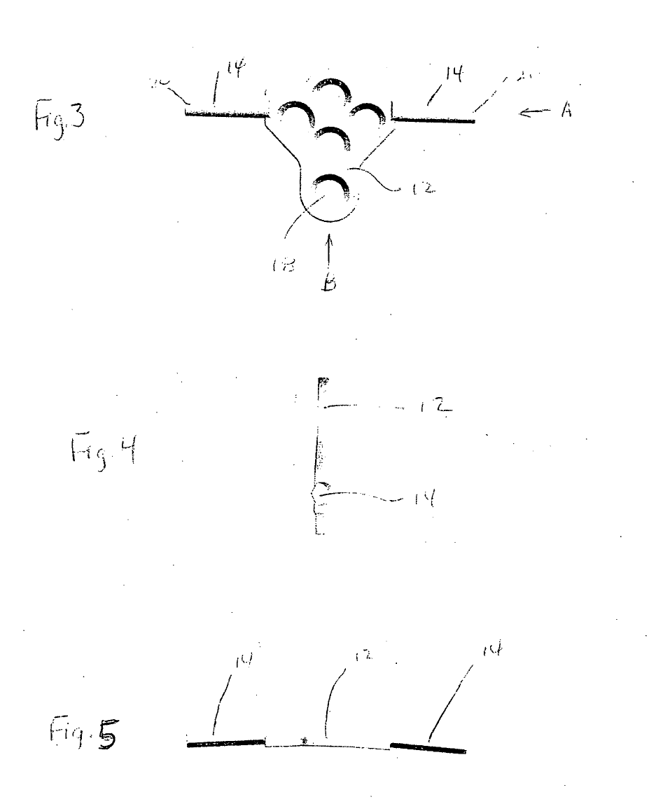

Referring now to FIG. 3, the center plate 12 is depicted in isolation, with the rod housings 16 removed. FIG. 4 shows the center plate 12, viewed in the direction of arrow A in FIG. 3. FIG. 5 is a view in the direction of arrow B in FIG. 3. As can be appreciated from the view of FIG. 5, the center plate 12 is curved at its bottom to fit the anatomy of the occiput. In other embodiments (not shown), the plate 12 is flat on the bottom. However, preferred embodiments employ curved center plates 12 for a better fit.

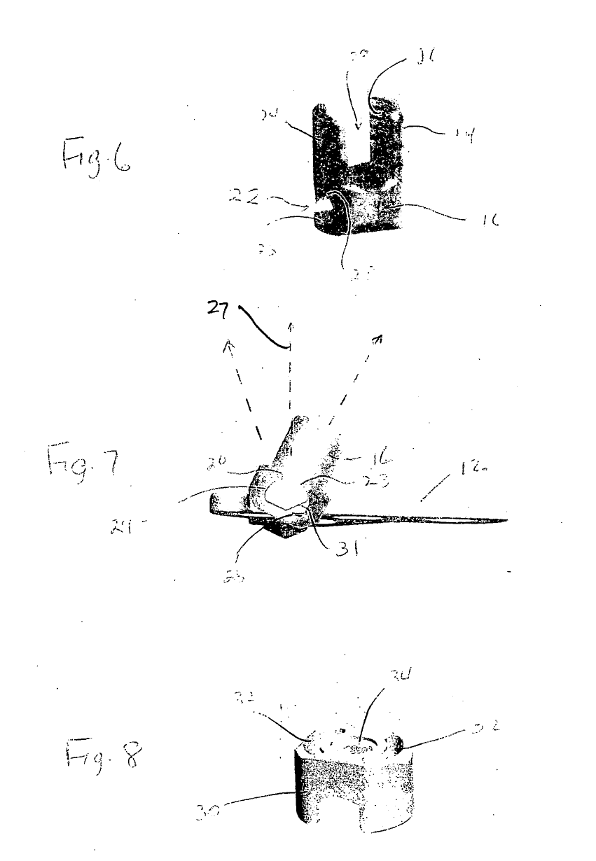

A perspective view of the rod housing 16 in isolation is provided in FIG. 6. The rod housing 16 includes a bore 22 through which an extension post 14 extends when the rod housing 16 is mounted on the center plate 12. Vertical extensions 24 extend upwardly and include threads 26. A rod channel 38 is formed between the vertical extensions 24. It is within this rod channel 38 that a fixation rod (or “stabilization rod”) is placed and held to the center plate 12. In the embodiment of FIG. 6, the bore 22 has an arcuate section 23 and flat section 25.

FIG. 7 is a side view of an assembled occipital plate assembly 10 in accordance with disclosed embodiments. In FIG. 7, the rod housing 16 is depicted in an angular position with respect to a vertical position (indicated by arrow 27). The rod housing 16 is rotatable in a sagittal plane, which is represented by the plane of the paper. In certain embodiments, the rotation of the rod housing 16 in the sagittal plane is limited to approximately ±25° from the vertical position, as indicated by the arrows in FIG. 7.

The interaction of the rod housing 16 with the extension posts 14 may be best appreciated in FIG. 7. The extension post 14 has an arcuate surface 29 and a pair of flat surfaces 31, when seen in cross-section and in an end view. The rod housing 16 is able to rotate on the arcuate surface 23 of the bore 22, riding the arcuate surface 29 of the extension post 14. The extent of the rotation of the rod housing 16 in the sagittal plane is limited by the interaction of the flat surface 25 of the bore 22 of the rod housing 16 with the flat surfaces 31 of the extension post 14. The flat surfaces 31 of the extension post 14 therefore act as rotational limit surfaces. The configuration of the extension post 14 and the bore 22 limit the range to approximately ±25° from vertical in the disclosed embodiment. However, in other embodiments, the range may be made greater or smaller than ±25°. By not providing a completely circular extension post 14, a lower profile for the assembly is achieved. Alternately, in certain embodiments, the extension posts 14 are completely circular so that the rod housings 16 may rotate a complete 360° around the extension posts 14. In each of the different embodiments, the bore 22 is appropriately configured to provide the desired range of motion when interacting with the extension post 14. In still other embodiments, the flat surfaces 31 are arranged so that the arc of rotation is relatively greater in either the caudal direction or the cephalad direction.

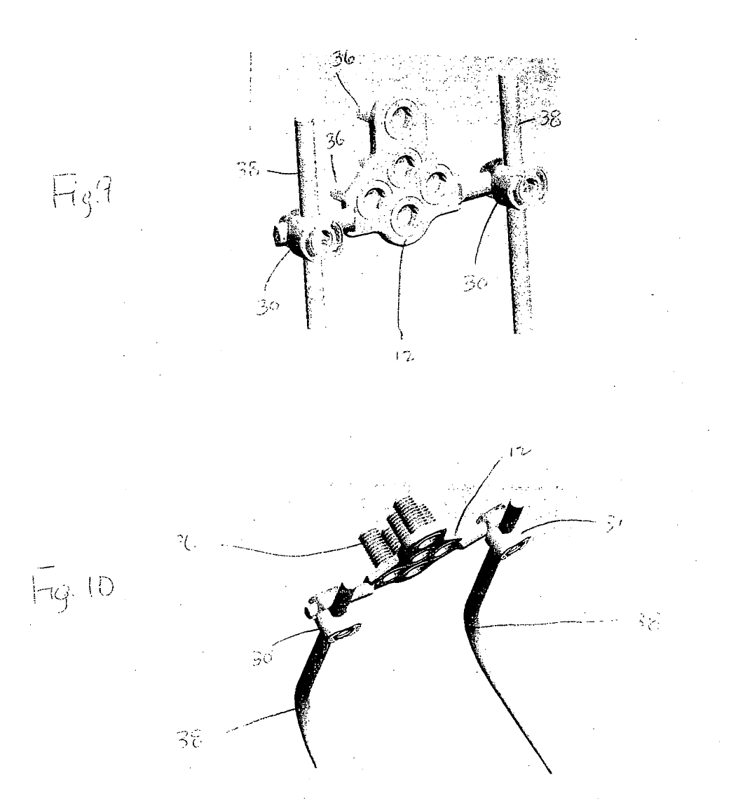

A locking cap is depicted in FIG. 8, which may be used with the occipital plate assembly 10 of FIGS. 1-7. The locking cap 30 includes slots 32 that fit over the vertical extensions 24 of the rod housing 16, after a fixation rod has been placed in the rod channel 38. An internal screw fastener 34 in the locking cap 30 is then turned, with the threads of the screw fastener 34 engaging the threads 26 in the rod housing 16, pulling the locking cap tighter against the rod and the rod housing 16. The interaction of the screw fastener 34 with the rod housing 16 acts to tighten the fixation rod to the center plate 12. Since the rod housings 16 are directly on the extension posts 14, a stronger and more direct locking of the rods on top of the extension posts 14 are provided, rather than if the rod housings 16 were not directly positioned over the extension posts 14.

FIGS. 9 and 10 provide different views of the occipital plate assembly 10 after the fixation rods 38 have been locked into place to the center plate 12. Occipital screws 36 are depicted extending through the holes 18 in the center plate 12. In an actual operation, the occipital screws 36 would be implanted into the occiput to secure the center plate 12 to the occiput.

The materials employed in the occipital plate assembly may be any suitable material, such as titanium, titanium alloy, etc.

The slidable and angularly adjustable rod housings 16 provide greater flexibility to a surgeon in the implantation process. The slidability of the housings on extension posts 14 in the medial-lateral direction accommodates variation in distance between the rods 38. This allows the rods 38 not to be confined to a set width that is determined by a fixed width of fixation rod holding elements on the occipital plate assembly. Further, since the rod housings 16 are angularly adjustable, or rotatable, in the sagittal plane, accommodation is made for rods bent at varying angles. This eliminates the need for additional bending of the rods of the implant.

Although the disclosed embodiments have been described and illustrated in detail, it is to be clearly understood that the same is by way of illustration and example only and is not to be taken by way of limitation, the scope of the invention being limited only by the terms of the appended claims.

What is claimed is:

1. An occipital plate assembly comprising:
a center plate configured for connection to a skull;
extension posts extending outwardly from the center plate; and
a rod housing rotatably mounted on each of the extension posts, the rod housings having an opening configured to receive a rod.

2. The assembly of claim 1, wherein the rod housings are rotatable in a sagittal plane.

3. The assembly of claim 2, wherein the rod housings are slidable along the extension posts in a transmedial-lateral direction.

4. The assembly of claim 3, further comprising a retaining element at a distal end of each extension post, the retaining elements maintaining the rod housings on the extension posts.

5. The assembly of claim 4, wherein each retaining element is a pin extending radially from one of the extension posts.

6. The assembly of claim 2, wherein the rod housings are limited in rotation in the sagittal plane to an approximately ±25° arc from a vertical position.

7. The assembly of claim 1, wherein the center plate is curved to accommodate occiput anatomy.

8. The assembly of claim 1, further comprising locking caps attachable to the rod housings and lockable to tighten rods inserted in the rod housings to the center plate.

9. The assembly of claim 1, wherein the center plate has holes configured for receiving occipital screws to secure the center plate to an occiput.

10. The assembly of claim 2, wherein the extension posts have a circular cross-section.

11. The assembly of claim 2, wherein the extension posts have a cross-section that has a semicircle and rotational limit surfaces that limit the rotation of the rod housings.

12. The assembly of claim 2, wherein the extension posts extend perpendicularly from the center plate in a transmedial-lateral direction.

13. The assembly of claim 2, wherein the extension posts extend at a non-perpendicular angle from the center plate.

14. The assembly of claim 3, wherein the rod housings each include a bore configured to slide on and at least partially rotate on one of the extension posts.

15. The assembly of claim 1, wherein the rod housings are mounted on the extension posts such that the openings of the rod housings are positioned directly over the extension posts in all angular positions of the rod housings with respect to the extension posts.

16. An occipital plate assembly comprising:
a center plate configured for attachment to an occiput; and
rod housings coupled to the center plate, the rod housings configured for securing fixation rods to the center plate and being angularly adjustable in a sagittal plane with respect to the center plate.

17. The assembly of claim 16, further comprising extension posts extending from the center plate, the rod housings being angularly adjustably mounted on the extension posts.

18. The assembly of claim 17, wherein the rod housings are slidably mounted on the extension posts and are slidable in a transmedial-lateral direction.

19. The assembly of claim 17, wherein each rod housing is configured to receive a locking cap that interacts with the rod housing to tighten a fixation rod to the center plate.

20. The assembly of claim 17, wherein the rod housings are angularly adjustable over an approximately ±25° range from a vertical position with respect to the center plate.

21. The assembly of claim 17, wherein the rod housings are angularly adjustable from a vertical position such that a range of motion is greater in a caudal direction than in a cephalad direction.

22. The assembly of claim 17, wherein the rod housings are angularly adjustable from a vertical position such that a range of motion is greater in a cephalad direction than a caudal direction.

23. The assembly of claim 17, wherein the rod housings are directly over the extension posts.
